(19) United States
(12) Patent Application Publication
(10) Pub. No.: US 2010/0081873 A1
(43) Pub. Date: Apr. 1, 2010

(54) SYSTEMS AND METHODS FOR OPTICAL VIEWING AND THERAPEUTIC INTERVENTION IN BLOOD VESSELS

(75) Inventors: Tetsuaki Tanimura, Acton, MA (US); Menahem Nassi, Palo Alto, CA (US); Mao Tanimura, Acton, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AiHeart Medical Technologies, Inc., Acton, MA (US)

(21) Appl. No.: 12/495,606

(22) Filed: Jun. 30, 2009

Related U.S. Application Data
(60) Provisional application No. 61/101,609, filed on Sep. 30, 2008.
(60) Provisional application No. 61/101,605, filed on Sep. 30, 2008.

(51) Int. Cl. A61B 1/04; A61M 29/02

(52) U.S. Cl. 600/109; 604/103.02

(57) ABSTRACT

An angioscope comprises a tubular sheath and a central member. The central member carries a lateral reflector for receiving images circumscribing the central member. The tubular sheath includes a light source for axially illuminating a vascular region as it is being optically imaged using the lateral reflector of the central member. The central member can be axially translated through the field illuminated by the light source on the tubular sheath. The angioscope may be combined in a catheter a catheter capable of delivering a therapeutic intervention while viewing a delivery site within a body passageway.

### CROSS-REFERENCES TO RELATED APPLICATIONS

The present application claims the benefit and priority of U.S. Provisional Patent Application No. 61/101,609 (Attorney Docket No. 027629-000200US), filed on Sep. 30, 2008 and entitled “Systems and Methods for Optical Viewing of Blood Vessels,” and U.S. Provisional Patent Application No. 61/101,605 (Attorney Docket No. 027629-000300US), filed on Sep. 30, 2008 and entitled “Methods and Apparatus for Intravascular Therapeutic Intervention Under Optical Visualization” the complete disclosures of which are expressly incorporated herein by reference.

### BACKGROUND OF THE INVENTION

1. Field of the Invention

The present invention relates generally to medical devices and methods. More particularly, the present invention relates to methods and apparatus for performing angioscopy and treating vulnerable plaque and other lesions in blood vessels, particularly the coronary vasculature.

Angioscopy refers to the direct optical viewing of blood vessels using an intravascular instrument, commonly referred to as an angioscope. The angioscope comprises a small diameter instrument capable of being advanced through the target vasculature and carrying both a viewing element and an illumination source. The viewing element typically comprises a fiberoptic bundle, but more recently might comprise a CCD or other miniature camera. The illumination source will also typically comprise fiberoptic fibers, but more recently could comprise a miniature LED or other illumination source. The use of angioscopes is advantageous in that it can provide real time color images of the vascular wall. Other technologies, such as intravascular ultrasound (IVUS) and optical coherence tomography (OCT) provide cross-sectional views of the blood vessel wall and surrounding tissue, but cannot provide color images of the wall surface itself. Such color wall images can be very useful in determining the nature of the vascular disease such as thrombus, yellow plaques, and mural hemorrhage which may be present.

Despite their promise, angioscopes are not commonly used in routine clinical settings because of certain limitations of available angioscopic catheter systems. One of the limitations is that most angioscopes are forward viewing, i.e. are configured to view axially from the distal tip. Such axial views do not provide detailed images of the lateral sidewall, and lesions on the sidewalls of blood vessels are viewed with difficulty and imprecision. Thus, it would be desirable to provide improved side viewing angioscopes capable of providing detailed images.

Certain side view angioscopes have been proposed. Most such side viewing angioscopes, however, have a limited viewing angle and utilize a mirror or prism for both illumination and viewing. Thus, while workable, such angioscopes must be rotated about their axes in order to provide a complete annular view of a region of a vascular wall.

Some side viewing angioscopes do have the ability to laterally image over a full 360° viewing angle, but the image is often disturbed and the illumination continues to be problematic. For example, as shown in U.S. Pat. No. 6,582,359, an angioscope has a single prism which both captures the image and illuminates over the viewing field. The use of a single prism for both illuminating and imaging, however, is problematic since the design is more complex and illumination in the direction of viewing often fails to provide the ability to distinguish surface contours as commonly associated with side illumination.

Forward and side-viewing angioscopes are also limited in their ability to combine their diagnostic capabilities with a therapeutic mechanism. Existing angioscopes do not provide for a combination of the advantages of a side-viewing mechanism, including direct and focused visualization of vessel wall structures without the blurring effect arising from out-of-focus and overlapping light reflection characteristics of forward-looking angioscopy, and a mechanism for delivering a therapeutic intervention. For instance, most angioscopes are only designed to approximate the location of the lesion, and their ability to contemporaneously target and deliver a therapeutic agent is limited. Moreover, the ability of angioscopes to distinguish types of plaque, particularly to identify vulnerable plaque, has also been limited.

For these reasons, it would be desirable to provide improved angioscopes and methods for angioscopic illumination and viewing of the vasculature, particularly diseased regions within the vasculature. Such apparatus and methods would desirably provide illumination at an angle different from the viewing angle in order to improve the topographic and contour detail provided by the image. In addition, it would be desirable if the position of the illumination could be adjusted relative to the viewing element of the system in order to allow a physician to adjust or improve the image produced. It would be still further desirable to provide imaging apparatus and modalities which are compatible with diagnosing particular types of coronary and other vascular lesions and for delivering needed therapies, such as drug delivery and photodynamic therapy, to such plaques after they have been diagnosed and identified. For example, it would be desirable to provide a side-viewing angioscope that is also capable of delivering one or more therapeutic interventions to enable the walls of the blood vessels to be viewed in real time the determine the location and nature of the lesion and contemporaneously provide the appropriate treatment without having to exchange the angioscope for a separate. Such combined devices should be easy to use, have a profile capable of being comfortably introduced into even the smaller coronary arteries, and preferably be compatible with the guidewire in a rapid exchange model. At least some of these objectives will be met by the present invention.

2. Description of the Background Art

U.S. Pat. No. 6,582,359 shows an angioscope with a prism arrangement which directs illumination radially outwardly and collects light back and directs the collected light back through a central wave guide structure. U.S. Pat. No. 6,887,196 describes an omnidirectional endoscope with a convex mirror for providing an annular viewing field and a circumferential light source for illuminating the viewing field. Other pertinent patents and published applications include U.S. Pat. No. 7,426,409; U.S. Pat. No. 7,250,041; U.S. Pat. No. 6,878,107; U.S. Pat. No. 6,817,976; U.S. Pat. No. 6,741,884; U.S. Pat. No. 6,706,004; U.S. Pat. No. 6,638,246; U.S. Pat. No. 6,537,209; U.S. Pat. No. 6,458,096; U.S. Pat. No. 6,450,950; U.S. Pat. No. 6,346,076; U.S. Pat. No. 5,976,076; U.S. Pat. No. 5,782,751; U.S. Pat. No. 5,644,438; U.S. Pat. No. 5,651,366; U.S. Pat. No. 5,569,162; U.S. Pat. No. 5,263,928; U.S. Pat. No. 5,078,681; U.S. Pat. No. 4,949,706; U.S. Pat. No. 4,784,133; U.S. Pat. No. 4,747,661; U.S. Pat. No. 4,471,779; U.S. Pat. No. 4,445,892; U.S. Pat. No. 4,213,461; U.S. Pat. No. 3,818,902; U.S. Pat. No. 3,773,039; US 2007/276184; US 2007/203396; US 2006/241493; US 2005/168616; US 2005/085698; US 2004/249247; US 2004/009044; and US 2002/103420, some of which are discussed further below.

Mackin, in U.S. Pat. No. 4,784,133, describes a ‘working well’ angioscope whereby the distal tip of a balloon is shaped such that an imaging or therapeutic device (such as a laser-emitting fiber) can be deployed against the tissue. Similarly, Trauthen et al., in U.S. Pat. No. 5,263,928, describe a catheter system which provides forward looking endoscopic visualization distal to an occlusive balloon. Hussein et al., in U.S. Pat. No. 4,445,892, disclose side-viewing optics in a balloon catheter system. However, this system uses two balloons to create an optically clear, flushed region between them; additionally, the device that is described by Hussein would likely experience guidewire interference in a rapid exchange model. Teamey et al., in U.S. Pat. No. 6,706,004 teach the use of a controlled balloon to achieve a precise clearance between the balloon and the lumen wall and requires use of optical coherence ranging to measure the distances. U.S. Patent Application No. 2004/0093044, to Rychnovsky et al., describes a fully occlusive balloon catheter with elements allowing flushing and therapeutic illumination distal to the balloon. Freeman et al., in U.S. Pat. No. 6,741,884, describe a probe designed for use in the infrared spectrum which utilizes special fluids that transmit infrared light in wavelength regions where typical fluids such as saline exhibit high absorption. One conventional method of delivering a therapeutic intervention is drug delivery through a balloon possessing micro-needles as disclosed in U.S. Pat. No. 6,638,246.

### BRIEF SUMMARY OF THE INVENTION

The present invention provides improved angioscopes and angioscopic imaging methods. The angioscopes provide for wide angle annular viewing of the interior of the blood vessel wall, typically with at least a 180° field of view and frequently with a full 360° field of view. The angioscope further provides enhanced illumination in a direction which is at an angle relative to the viewing angle, typically at a right angle, but optional, at an oblique or acute angle as well. In some instances, it may be possible to adjust the relative angle to improve the discernment of contour detail and/or color.

In the exemplary embodiments, the illumination is provided in an axial direction from a sheath surrounding an imaging core. The sheath typically includes a plurality of illumination elements over its distal end, and the imaging core provides for lateral imaging over a wide angle annular view, where the annular vessel wall section is illuminated by the axial illumination. By imaging in a radial direction and illuminating in an axial direction, the light will reveal contours and other detailed structures of the vessel wall which would not be as apparent with both imaging and illuminating in a radial direction. Moreover, by allowing the imaging core to axially translate relative to the illuminating sheath, the illumination can be further adjusted relative to the viewing angle in order to modify and enhance the image at any particular region under scrutiny. The angioscopes and angioscopic systems of the present invention are particularly useful for imaging in the visible light range. The systems may further comprise red light pass filters for imaging and detecting thrombus and yellow light pass filters for imaging and detecting lipids and plaque.

In a first aspect of the present invention, an angioscope for optically imaging a luminal wall comprises a tubular sheath and a central member comprising having an image viewing element at its distal end and a lateral reflector disposed distally of the image viewing element to gather the image and transmit it to the image viewing element. The tubular sheath has a proximal end, a distal end, a central lumen, and a light source disposed at the distal end to direct light axially from the sheath. The central member is reciprocatably received in the central lumen of the tubular sheath and has a proximal end and a distal end. The image viewing element is disposed near the distal end of the central member, and the lateral reflector is disposed distally of the image viewing element to reflect the image back to the viewing element. Thus, the tubular sheath forms one component of the angioscope while the central member, image viewing element, and lateral reflector will typically be joined together in a fixed geometry to provide a second component or assembly, referred to herein as the imaging core. The imaging core is reciprocatably mounted within the tubular sheath, providing a number of advantages. In addition to the ability to adjust the position of the viewing element relative to the illumination source, the use of a smaller diameter imaging core allows the imaging core to be advanced into regions of the vasculature which are smaller than the tubular sheath. Moreover, the annular region between the tubular sheath and the exterior of the imaging core allows for the introduction of saline or other clear viewing media to provide the clear field necessary for optical viewing in a blood vessel.

In certain exemplary aspects of the angioscope, the light source will comprise at least one fiberoptic element disposed axially in a wall of the tubular sheath, typically comprising a plurality of fiberoptic elements (bundles and/or fibers) circumferentially spaced-apart over a portion or all of the distal end of the tubular sheath. Alternatively, the light source could comprise one or more light emitting diodes (LEDs), usually a plurality of LEDs circumferentially spaced apart over the distal end of the tubular sheath.

The image viewing element will typically comprise a fiberoptic bundle having a distal surface for receiving the image of the blood vessel wall. Optionally, a lens may be provided in order to focus the optical image into a distal end of the fiberoptic bundle. Alternatively, the image viewing element may comprise a CCD (charge coupled device) or other solid state camera located near the distal end of the central member for receiving the blood vessel wall image. In either case, the lateral reflector will comprise a reflective element disposed to reflect an image surrounding an annular region of the central member back to the image viewing element. The reflective element may be a single flat reflective surface for reflecting a limited angle of the wall surface, typically in the range 90° to 100°. Usually, however, the lateral reflector will be adapted to deliver an image over a circumferential viewing angle of at least 180° about the axis of the central member, more typically being over a full 360°. The lateral reflector may comprise multiple flat surfaces disposed to reflect images from the circumferential arc, usually including at least three reflective surfaces, and often including four or more reflective surfaces. Alternatively, the reflective element may comprise a partial or full conical prism to reflect a continuous image spanning a circumferential arc surrounding the central member. In all cases, the circumferential arc may extend fully around the central member, i.e. over a full 360°.

The angioscope may be configured for delivery through the lumen of an angioplasty or other catheter. Alternatively, the angioscope may be configured for delivery over a guidewire, where the guidewire lumen may be disposed in the tubular sheath, optionally being disposed along one side of a distal section of the sheath in the manner of a “rapid exchange” catheter.

Optionally, the angioscopes may comprise inflatable occlusion members, typically inflatable balloons, near the distal ends of the tubular sheaths. The occlusion members usually comprise an elastomeric balloon or cuff which circumscribes a distal portion of the sheath body. The occlusion member may be inflated using a pressurized source connected via lumens formed within the sheath, but will preferably be self-inflating through a plurality of ports in the wall of the tubular sheath where the ports are configured to allow infusion medium flowing through the lumen of the sheath to pass through the ports and inflate the balloon. When the flow of infusion medium is stopped, the balloons will deflate and collapse over the exterior surface of the balloon due to their elasticity.

In a second aspect of the present invention, methods for viewing the wall of a blood vessel comprise introducing a tubular sheath into a lumen of the blood vessel. The wall of the blood vessel lumen is illuminated in an axial direction from a light source on or near a distal end of the sheath, where the light source may have any of the configurations described previously. A central member is advanced from the central lumen of the sheath and includes a surface for reflecting an optical image of the vessel wall to a viewing element on the central member. The reflective surface may have any of the structures described previously for a lateral reflector, while the viewing element may comprise an optical fiber bundle, a CCD camera, or any other conventional imaging device capable of collecting light and either converting light directly into an electronic signal or delivering the light through the central member to an external camera or similar element for converting the light to an electronic signal. In all cases, the optical image will be transmitted to a viewing screen to provide a real time image of the blood vessel wall.

The viewing methods of the present invention may optionally comprise inflating an occlusion member which circumscribes a distal portion of the tubular sheath. Inflation typically comprises diverting a portion of an infusion medium which flows through the tubular sheath, typically the medium which clears the blood vessel to permit optical viewing. The occlusion member typically comprises an elastic balloon or other cuff-like structure, as described above, which inflates when infusion medium flows through the sheath, typically through a plurality of ports formed in the sheath wall. The elastic balloon will deflate and collapse over the sheath when the infusion medium stops flowing through the sheath. Forming an occlusion surrounding the sheath reduces or stops blood flow past the distal end of the sheath, further clearing the viewing region of blood and facilitating optical viewing of the vascular wall. Such self-inflating occlusion members and their use may also find utility with the treatment embodiments of the present invention, as described below. For example, occluding blood flow temporarily using the occlusion balloons could enhance drug flow delivery to the region distal to the balloons, optionally using drugs carried by an infusion medium.

The present invention also provides a combined viewing and treatment catheter capable of viewing a target site within a body passageway and delivering therapeutic intervention to the target location either while or immediately following such viewing. The combined viewing and treatment catheter comprises a catheter body having a proximal end, a distal end and multiple lumens there-between. A mechanism configured to deliver a therapeutic intervention such as an angioplasty balloon, a porous balloon for delivering drugs into the vascular wall, a needle for delivering drugs, an electrode for delivering energy, a blade for atherectomy, or the like, is disposed at the distal end of the catheter body, and the distal end of the catheter carries a side-viewing mechanism that allows location and evaluation of a diseased region of the vessel wall prior to the therapeutic intervention. The catheter carries a plurality of optical fibers in optical communication with the side viewing mechanism, typically through a lumen in the catheter body, and at least one lumen in communication with the mechanism configured to deliver a therapeutic intervention.

A side-viewing mechanism of the combined viewing and treatment catheter comprises an imaging lens and a beam director. Additionally, the device comprises optical fibers that transmit electromagnetic radiation of a predetermined wavelength range, such as visible light, flowing bi-directionally between the proximal end and distal end of the catheter to illuminate and visualize the target site.

The combined imaging and treatment catheter could be used to treat occlusions or lesions (such as thrombus and plaque) of the body passageway. The therapeutic intervention delivery mechanism could include an angioplasty balloon, a multi-lumen balloon with a porous exterior or other drug-delivery balloon, a therapeutic injection needle, a stent-delivery mechanism, a thrombectomy device, a thrombus aspiration device, or the like. The catheters are particulary useful for treating vulnerable plaque, for example by delivering laser energy to ablate a particular type of lipid plaque, referred to as yellow plaque, which can be readily identified using the optical viewing system of the present invention. The system is also particularly useful for delivering light at a specific wave length for performing photodynamic therapy and other treatments where a particular drug or chemical entity can be activated by the light, for example to inhibit smooth muscle cell proliferation in order to stabilize vulnerable plaques.

### BRIEF DESCRIPTION OF THE DRAWINGS

FIG. 1 illustrates a system constructed in accordance with the principles of the present invention which includes an angioscope and a coupled viewing screen;

FIG. 2 illustrates the distal end of the angioscope of FIG. 1, illustrating the tubular sheath and the central member therein;

FIG. 2A illustrates an alternative embodiment of the distal end of the angioscope of the present invention;

FIG. 2B illustrates an embodiment of the present invention where the tubular sheath includes a self-inflating elastic occlusion member;

FIG. 3 is an alternative view of the distal end of the angioscope of the present invention, illustrating a tubular sheath having a guidewire lumen;

FIG. 4A-4C illustrate the use of the system of FIG. 1 for obtaining a real time, optical image of a diseased region in the vasculature;

FIG. 5A illustrates one embodiment of a multi-lumen catheter in accordance with the present invention;

FIG. 5B shows an isolated detailed view of a portion of an embodiment, namely a side-viewing mechanism;

FIG. 5C shows an isolated detailed view of a portion of an embodiment, namely a delivery mechanism for a therapeutic intervention;

FIG. 6 illustrates another embodiment of the present invention;

FIG. 7A shows the in vivo operation of one embodiment of the side viewing angioscope; and

FIG. 7B shows a therapeutic intervention introduced through a balloon comprising micro-needles.

### DETAILED DESCRIPTION OF THE INVENTION

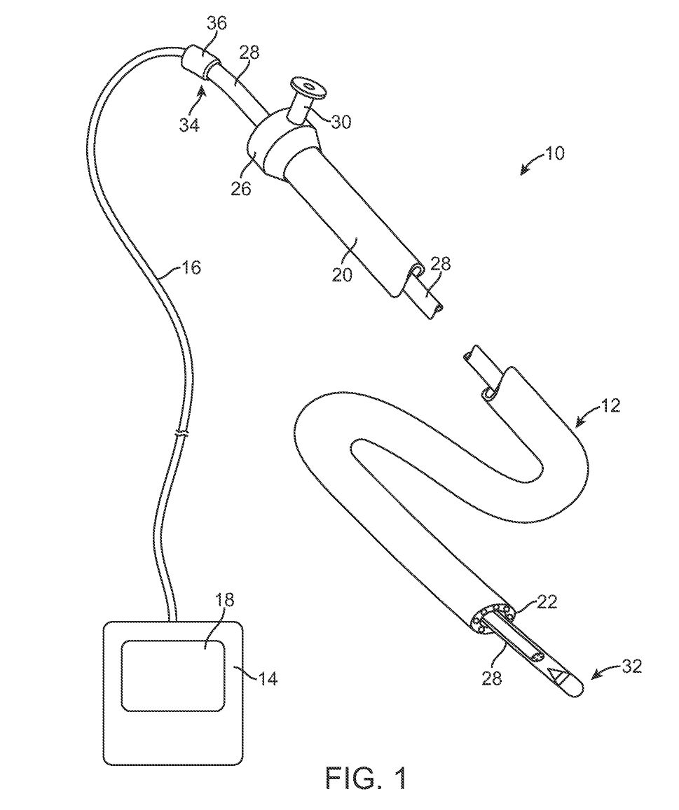

A side view angioscopy system 10 constructed in accordance with the principles of the present invention includes an angioscope 12 connected to a viewing console 14 by a cable 16 is illustrated in FIG. 1. The viewing console 14 will include a video display 18 and the electronics necessary to convert an electrical signal from a CCD camera or other video conversion element to an image which can be shown on the display 18. As will be described in more detail below, the CCD camera or other video element may be included as part of the angioscope or alternatively could be included within the video console 14 itself (where the image would be carried to the CCD via optical fibers in the cable 16).

The angioscope 12 includes a tubular sheath 20 having a distal end 22 and a proximal end 24. A connecting hub 26 is provided at the proximal end 24 of the sheath and includes a hemostatic valve (not shown) which reciprocatably receives a central member 28. Usually, at least one additional port 30 will be provided on the hub 26 to permit access to the central lumen of the sheath. The port 30 may be used, for example, for delivering saline or other clear fluid in order to provide a clear visual field for viewing with the angioscope, as will be described in more detail below. The central member 28 also has a distal end 32 and a proximal end 34. A hub or connector 36 is provided at the proximal end 34 of the central member 28.

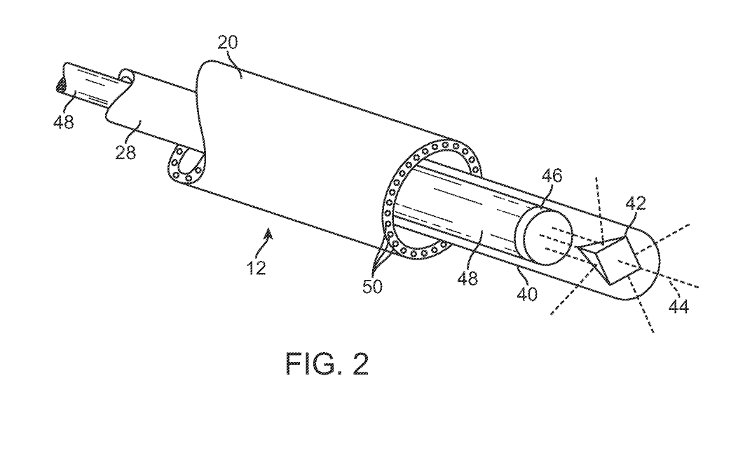

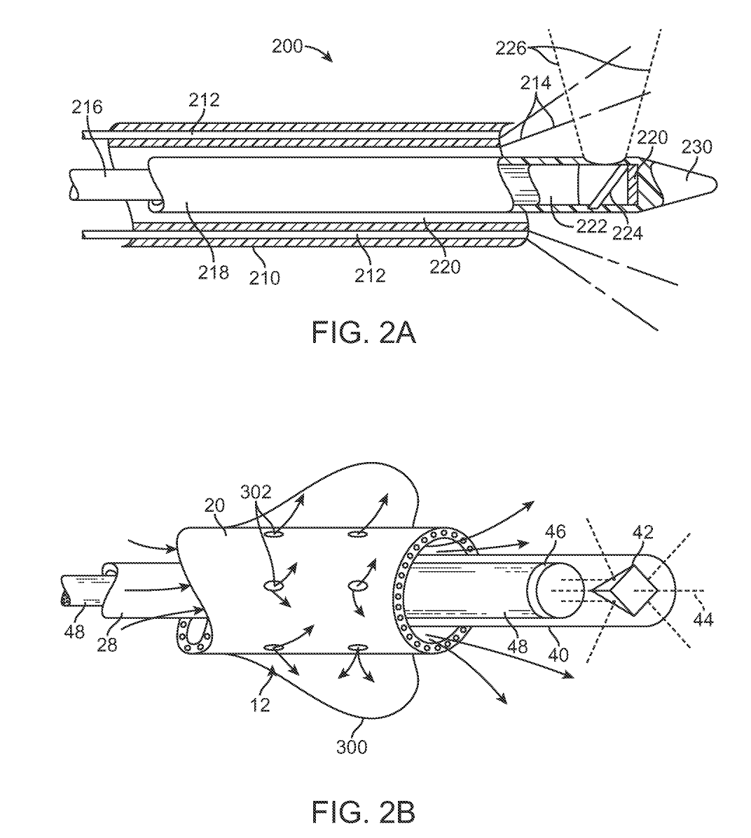

Referring now to both FIG. 1 and FIG. 2, the central member 28 comprises an outer sleeve 40 which is typically transparent, at least over its distal end where the imaging components are located. The imaging components include a lateral reflector 42 which is illustrated as a square pyramid capable of receiving light images from the four orthogonal directions emanating radially from the axis 44 of the central member. The pyramid may have mirrored faces, but will more typically be a prism capable of reflecting the orthogonally originating images to an axial direction so that they enter a lens 46 which is disposed at the distal end of a fiberoptic bundle 48. The light images are thus reflected and focused into the fiberoptic bundle 48 and are transmitted to the proximal end of the central member 28 where, optionally, a CCD camera may be disposed in the hub 36. The CCD camera converts the optical light images into electrical signals which may be carried by cable 16 to the viewing scope 14 for display on screen 18. Alternatively, but not illustrated, the CCD camera could be located in the distal region of the central member 28 in order to receive the optical images directly from the lateral reflector 42. In that case, an electrical signal generated by the CCD would be carried down the length of the central member by wires.

A particular feature of the present invention lies in the axial delivery of light from the distal end of the tubular sheath 20. While the light could be provided by an array of light emitting diodes (LEDs) at the distal end of the sheath, more typically it will be provided by a plurality of optical fibers 50 which are disposed in the wall of the tubular sheath. The optical fibers 50, in turn, receive light from an illumination source typically disposed in the hub 26. Although not shown, a power cord will typically be provided to the hub in order to provide power to the light source.

The tubular sheath 20 will have dimensions suitable for intravascular delivery to a desired target site, typically within the coronary vasculature. Thus, the sheath 20 will typically have a diameter in the range from 1 mm to 3 mm, usually being from 1.3 mm to 2 mm. The sheath 20 will have a length in the range from 100 cm to 200 cm. The wall thickness of the sheath will be sufficient to hold the optical fiber 50, typically having a thickness of about 0.1 mm. The optical fibers will typically have a diameter less than 0.05 mm, and usually from 30 to 40 fibers will be provided in the sheath.

The central member 28 will have a much smaller diameter than that of the tubular sheath 20, typically having a diameter of 1 mm or less, usually being from about 0.4 mm to about 0.8 mm. The fiberoptic bundle 48 within the sleeve 40 of central member 28 will usually have a diameter only slightly less than the central member. This spacing between the outer sleeve 40 of the central member and the inner wall of the sheath 20 will usually be from 0.1 mm to 0.2 mm, allowing the introduction of saline, contrast media to flush the blood vessel distal to the tip of the sheath 20 to permit angioscopic viewing distal to the sheath within a field of view of the lateral reflector 42 on the central member 28. The lens 46 will typically be a GRIN lens which focuses light through a precisely controlled radial variation of the lens material's index of refraction from the optical axis to the edge of the lens, providing a focusing depth between 1 mm to 2 mm. The pyramid prism will typically have a base diameter with dimensions in the range from 0.2 mm to 0.8 mm, with sides converging at an angle in the range from 30° to 70°, typically being 45°. The materials are designs suitable for the GRIN lens and prism are well known in the micro-optics component industry, including details of injection molding and other fabrication techniques.

A specific angioscopy system 200 is illustrated in FIG. 2A. The angioscopy system 200 includes an outer sheath 210 having a plurality of optical fibers 212 formed in its wall, generally as shown above in FIG. 2. The sheath 210 can be formed, for example, from a non-distensible polymer, such as polyethylene terephthalate (PET) having a narrow thickness, typically about 0.1 mm or less. The light fibers 212 will typically have a diameter of about 50 μm. The distal ends of the light fibers will be oriented to provide a diverging light beam indicated by lines 214, where the beam typically diverges at an angle of about 10° outwardly toward the vascular wall. An image fiber bundle 216 will typically have a diameter of about 0.3 mm and include 3,000 pixels and be encased in a second tubular sheath 218, typically having a thickness of about 0.1 mm and being formed from PET. Thus, an annular lumen 220 will typically remain to provide for the flow of infusion medium.

The light fiber bundle 216 will terminate in a grin lens (0.3 mm) 222 which receives light reflected from the vascular wall which is reflected by a mirror 224 mounted at 45°. The lens and mirror are configured to provide for a viewing angle in the lateral direction which diverges at about 60°, as indicated by lines 226. Conveniently, at least one platinum marker is located just proximal of an atraumatic, typically elastomeric tip, 230.

Optionally, the angioscopy system of the present invention may include an inclusion element 300 which circumscribes a distal region of the tubular sheath 20, as shown in FIG. 2B. The angioscopy of FIG. 2B is identical in all respects to that illustrated in FIG. 2 except for the provision of the elastomeric occlusion member 300 and a plurality of inflation ports 302 formed in the wall of the tubular sheath and located so that they do not disrupt the light fibers therein. When the infusion medium 28 is flowing through the lumen of the tubular sheath 20, a portion of the medium will be diverted into the interior of the occlusion member 300, causing the occlusion member 300 to radially expand (inflate) as illustrated in FIG. 2B. When the flow of infusion medium 28 ceases, the elasticity of the occlusion member 300 will cause it to collapse over the outer wall of the tubular sheath so that the angioscopy system can be repositioned, withdrawn, or otherwise moved within the vasculature without interference from the occlusion member 300.

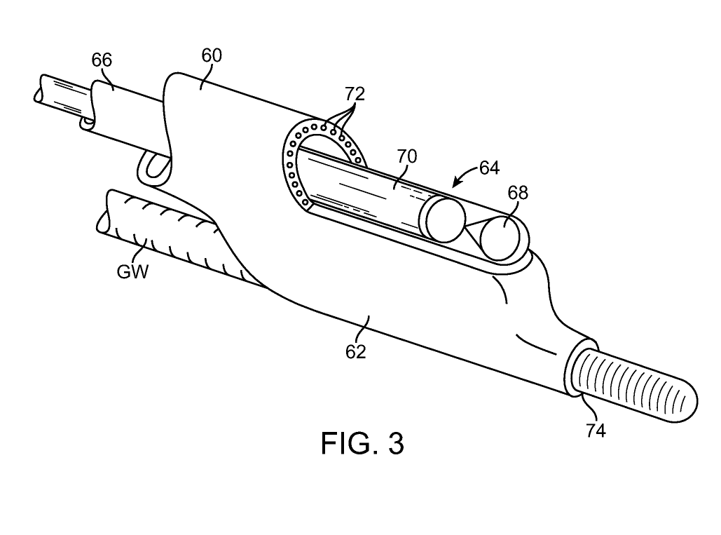

An alternative configuration of a tubular sheath 60 and central member 66 constructed in accordance with the present invention is shown in FIG. 3. The tubular sheath 60 includes a monorail section 62 for receiving a guidewire GW. A cutout region 64 disposed over the monorail section 62 receives the distal end of the central member 66. The central member 66 is constructed in generally the same manner as the central member 28 discussed above, except that a lateral viewing element 68 comprises a conical pyramid for receiving and reflecting light to a fiberoptic bundle 70. The use of a conical pyramid, which may be a mirror but more usually will be a prism, provides a more uniform 360° view circumscribing the central member.

A plurality of light fibers 72 may be provided in the wall of the tubular sheath 60 and will terminate in a generally vertical face of the cutout 64, as shown. Light source(s) could be provided at other regions of the distal end of the tubular sheath. For example, an LED source could be provided at the distal end above the guidewire port 74.

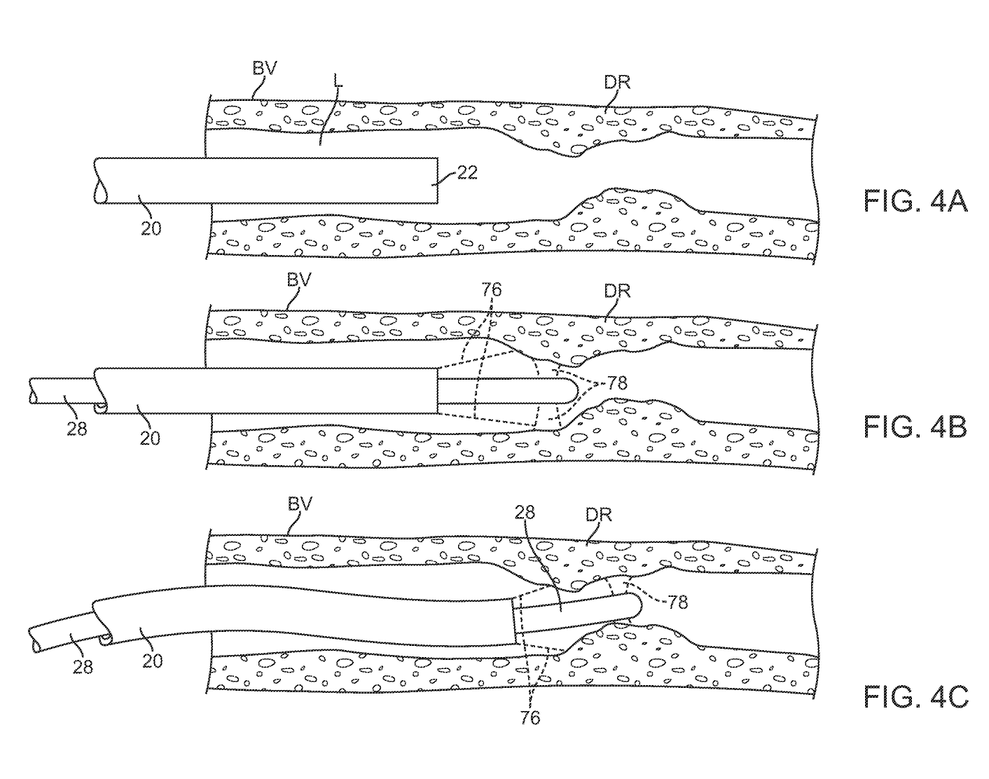

Referring now to FIGS. 4A-4C, use of the angioscopic system 10 for optical viewing of a diseased region DR in a blood vessel BV will be described. Initially, tubular sheath 20 is introduced to lumen L of the blood vessel BV in a conventional manner. Optionally, the sheath 20 may be introduced over a guidewire (not shown) which is then removed and exchanged for the central member 28. Alternatively, the tubular sheath 20 could be introduced together with the central member 28 through an external guide catheter.

Once the sheath 20 has reached the diseased region DR to be imaged, the central member 28 will be advanced from the distal end 22 of the sheath, as shown in FIG. 4B. As the central member 28 provides lateral viewing about its periphery, the central member will be advanced to within the diseased region DR, as shown in FIG. 4B. Axial illumination is provided by the optical fibers 50 in the sheath 20, as shown by broken lines 76. Viewing is performed in the lateral direction, as shown by viewing lines 78. It will be appreciated that axial illumination coupled with the lateral viewing will enhance the ability to observe the contours of the diseased region. Additionally, the axial illumination facilitates viewing as the central member is axially advanced and retracted, as shown in FIG. 4C. The use of the smaller central member 28 for viewing permits access to even smaller luminal regions than would be possible if the sheath were attached to the central member. Usually, saline or other clear fluid will be introduced through the lumen of sheath 20 during the view in order to provide a viewing field clear of blood.

The angioscopes of the present invention may be adapted or modified to deliver one or more therapeutic interventions. The angioscope is introduced into a blood vessel or other body passageway, usually a coronary artery, and a target site for an intended therapeutic intervention is visually determined in real time. Thereafter, the one or more therapeutic interventions are delivered to the target site using the same device, optionally while continuing to view the target site with the angioscope. While the present disclosure describes an angioscope for use in blood passageways, it should be noted that the angioscope may be incorporated into a larger catheter or other treatment system.

Exemplary combined viewing and treatment catheters may comprise side viewing angioscopes with multiple lumens, at least one of which terminates distally at a side-viewing mechanism and another of which terminates distally at a delivery mechanism of a therapeutic intervention. The side viewing mechanism may comprise a fiber-optic bundle and a prism. The fiber optic bundle(s) transmit visible light to the prism, which relfects the light through a transparent portion of the catheter wall and onto the blood vessel wall. Light reflected by the vessel wall is reflected again by the prism and is directed back along the same or different fiber optic bundles to an external console as described above. The images thus captured on the console are viewed by a user and used assist in locating the target site and identifying the nature of the lesion, so that the user can determine the appropriate treatment modality to treat that site.

After such determination is made, a therapeutic intervention is delivered to the treatment or target site. The delivery mechanism is usually located within the same catheter as the side-viewing mechanism. In one embodiment, the delivery mechanism is a balloon that is in fluid communication, through the lumen, with the proximal end of the catheter. In such an embodiment, the balloon is a drug delivery balloon comprising pores, micro-needles, or other suitable mechanisms for drug delivery. In another embodiment, the balloon is an angioplasty balloon. In another embodiment, the balloon is a stent-delivery balloon. In another embodiment, the delivery mechanism is a thrombectomy device or a thrombus aspiration catheter. Drug delivery needles may also be incorporated into the catheters.

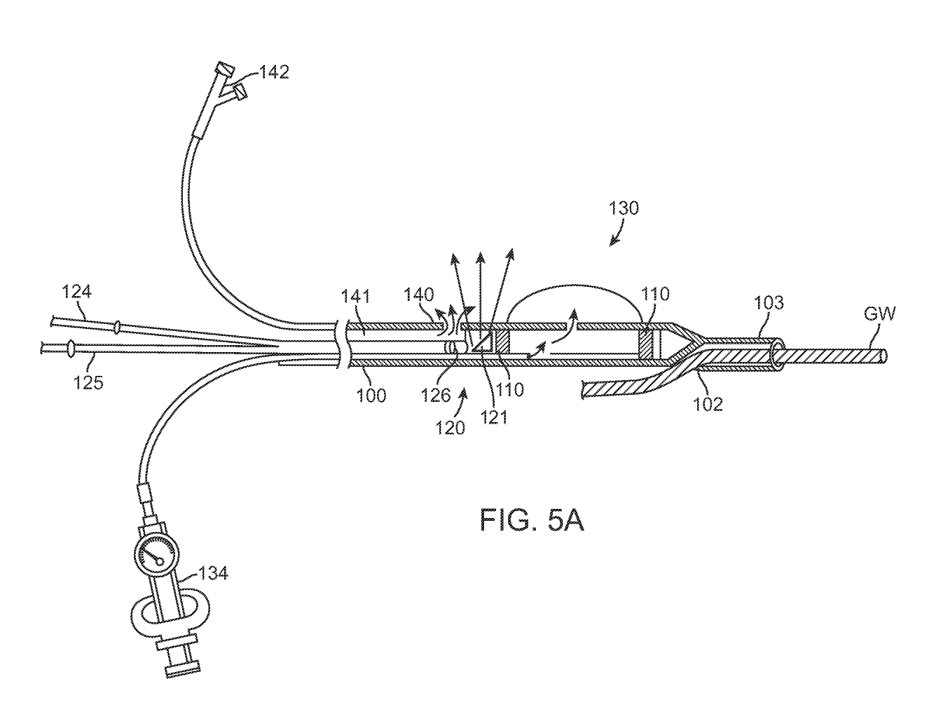

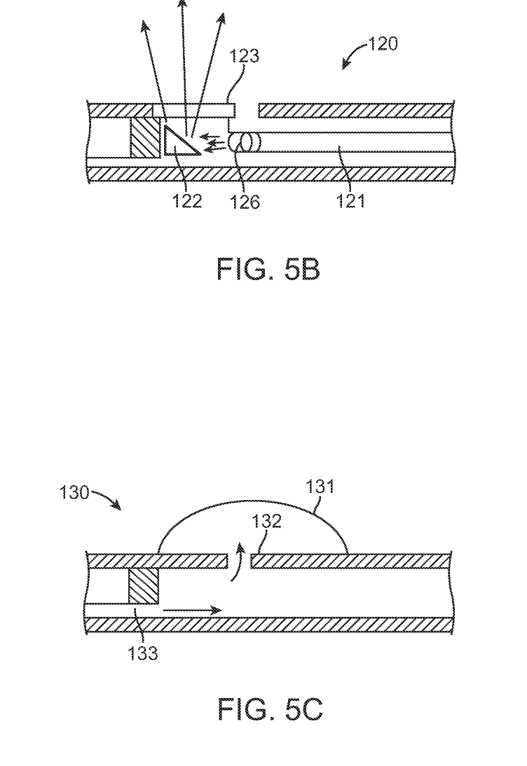

Turning now to FIG. 5A, a therapeutic delivery angioscope comprises a catheter body 100 having a proximal end, a distal end, and multiple lumens therebetween. The angioscope is introduced into the body passageway over a guidewire GW. The embodiment shown here is a rapid exchange catheter embodiment, wherein the guidewire GW is held within a guidewire lumen 103 in the distal tip of the catheter 100, and exits at a port 102. A lumen 141 of the catheter body 100 carries a side-viewing mechanism 120. This side-viewing mechanism 120 is in optical communication with a light input port 124 and an image receiving port 125 at the proximal end.

The catheter body 100 additionally comprises a therapeutic delivery mechanism 130 for delivering a therapeutic intervention, near the distal end of the catheter body 100. the delivery mechanism 132 is optionally in communication with a delivery input port 134 located at the proximal end of the catheter 100. Delivery input port 134 may be configured to allow a user to proximally introduce a medium into the catheter for delivery at the distal treatment site. Additionally or optionally, catheter 100 comprises a flushing port 140 which is connected to the lumen 141 that is in communication with a Y-body connector (not shown) at the proximal end of the catheter 100. Additionally or optionally, catheter 100 may comprise one or more radiopaque markers 110. In the embodiment shown in FIG. 5A, one marker 110 denotes the location of the balloon and another denotes the location of a viewing prism 122 of side viewing mechanism 120.

FIG. 5B shows the side viewing mechanism 120 in more detail. The side-viewing mechanism 120 comprises a fiber bundle 121 comprising a plurality of optical fibers that terminate in lens 126. An exemplary lens to be used is a GRIN lens as described above. The optical fibers in bundle 121 are in optical communication with a beam director, such as the prism 122, at the distal end. The optical fibers are configured to transmit electromagnetic radiation of a pre-determined length (e.g., visible light) flowing bi-directionally between the proximal end and distal end of the catheter. Additionally, the portion 123 of the catheter wall adjacent to the prism 122 is transparent to allow for the transmission of light rays. Alternatively, a portion of the catheter wall can be opaque such that no light is transmitted through the opaque portion of the catheter and only the transparent part of the catheter permits light transmission. The therapeutic catheters could also employ a light source in an external sheath as described above for other embodiments of this invention.

FIG. 5C shows a detailed view of one embodiment of the delivery mechanism 130 of the therapeutic intervention. In this embodiment, the mechanism 130 comprises a balloon 131. Balloon 131 is shown as a partially occlusive balloon, but could also be a fully occlusive balloon with appropriate accommodations for a guidewire in an over-the-wire catheter. Balloon 131 is configured to be an angioplasty balloon, a stent-delivery balloon, a drug delivery balloon, or any such therapeutic intervention. As shown in this embodiment, the balloon 131 is in fluid communication with the catheter shaft through opening 132, which is in turn in fluid communication with a second lumen 133. Lumen 133 is in communication with the external inflation port 134 (not shown in FIG. 5C). When inflated, the balloon 131 is configured to substantially contact at least some portion of the body passageway. When the balloon 131 is a drug delivery balloon, it comprises one or more microneedles (not shown) along the outer surface of the balloon. These microneedles facilitate drug delivery into the vessel wall by allowing penetration of the drug into the wall. Alternatively, the drug delivery balloon may comprise perforations or otherwise be porous along the outer surface of the balloon wall. Optionally, when the balloon is inflated, it occupies an angular region around the catheter body 100 that is less than 360 degrees to accommodate a guidewire, where at least some portion of the guidewire is located exterior to the catheter.

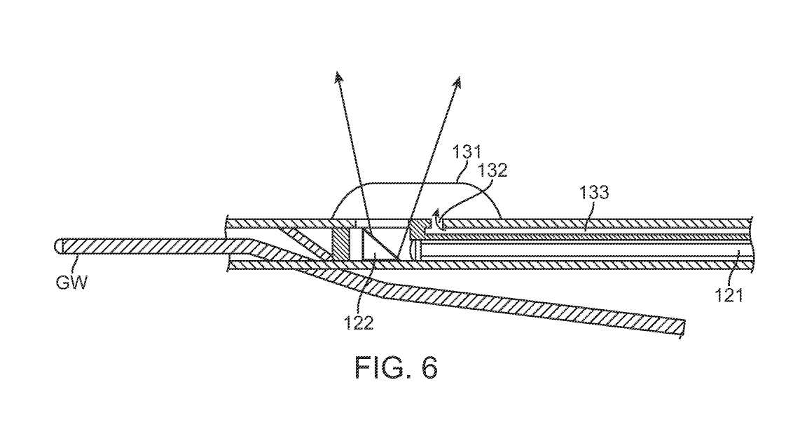

As shown in the previous figures, the balloon 131 is located distal to the prism 122 of the side viewing mechanism 120. Alternatively, as shown in FIG. 6, the balloon 131 could be located above the side viewing mechanism 120. In this embodiment, the balloon 131 would be made of transparent material, and any fluid entering the balloon would be of sufficient translucence as to maintain visualization by the side viewing mechanism 120.

A rapid exchange catheter embodiment, for example as shown in FIG. 2 described above, can be modified to include a treatment delivery mechanism For example, a user may introduce flushing media, therapeutic interventions, or any combination thereof through the cut out 64, either with the fiber optic bundle remaining or removed. Exemplary therapeutic interventions that may be introduced by this method are thrombus aspiration and fluid drugs. Additionally or optionally, the catheter wall surrounding cut out 64 is embedded with one or more optical fibers 72 which could be used to deliver laser light for therapy.

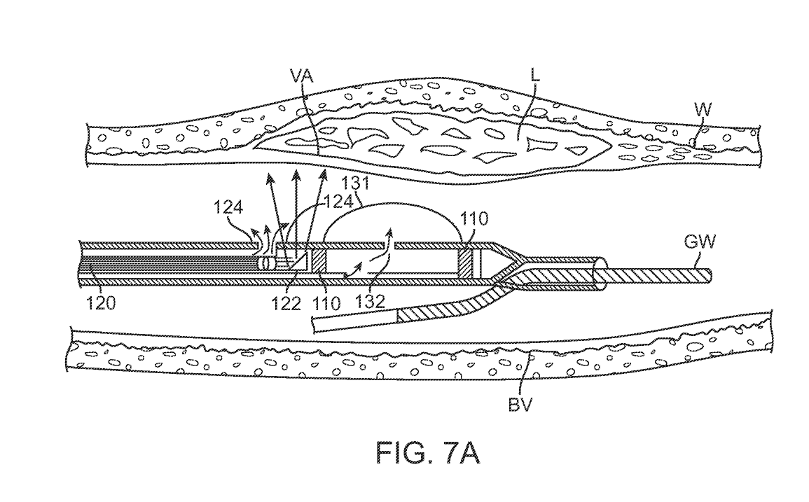

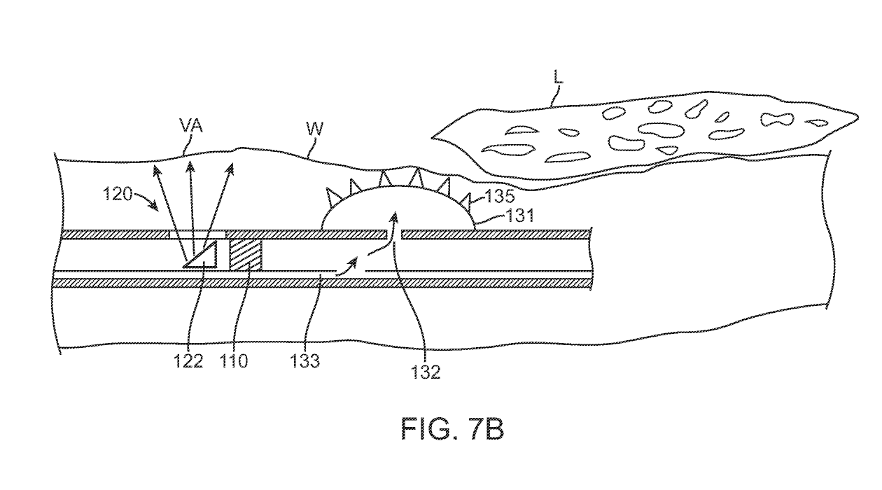

The in vivo operation of one embodiment of the side viewing angioscope is shown in FIG. 7A. The angioscope is introduced into a blood vessel BV using a guidewire GW and is advanced to a desired treatment site. Exemplary treatment sites include, but are not limited to, areas of partial occlusion, areas with thrombus, and previously stented areas. At the desired treatment site, the blood vessel wall W is viewed using the side viewing mechanism 120. The optical fibers 121 communicate light to the prism 122. The light is reflected off the prism 122, and travels through the transparent portion 124 of the catheter and onto a viewed area VA of the blood vessel wall. Light that reflects off the viewed area VA of the blood vessel wall is collected by the prism 122 and is transmitted back to the proximal end through another set of optical fibers contained within fiber bundle 121. The images thus captured are viewed by an external viewer. By moving the catheter 100 within the blood vessel BV and studying the resulting images, the site of the lesion L is identified. The term “lesion” is meant to include thrombus, plaques, any other occlusions and previously stented areas.

After a lesion has been identified, the appropriate specific treatment site for the lesion is determined. For example, if the therapeutic intervention to be introduced is a drug, an exemplary site for treatment and delivery of a therapeutic agent would be the area immediately proximal to the lesion site to allow the blood vessels (which are not found in the lesion site) to absorb and distribute the therapeutic agent. This is shown in FIG. 7B, where the drug (the therapeutic intervention) is to be introduced through a balloon 131 comprising micro-needles 135. This site immediately proximal to the lesion would be determined by positioning the prism 122, through forward and backward movement of the catheter. The user would assess the images of the viewing area VA captured on the monitor to determine the locations and dimensions of the lesions. Using the images thus captured, the user would position the catheter such that the viewing area VA is adjacent to the lesion. Simultaneously and optionally, the radiopaque markers 110 may be used to guide the location of the prism 122 and the balloon 131.

As another example, if the therapeutic intervention to be introduced is a stent or an angioplasty balloon, the appropriate site for delivery of the therapeutic agent is a central area of the lesion. In such a situation, the user would position the catheter such that the viewing area VA is directly within the lesion.

After the site of the treatment has been determined, the appropriate therapeutic intervention is introduced. As previously mentioned, in the embodiment shown in FIG. 7B, the therapeutic intervention comprises a drug-delivery balloon that is configured to deliver a therapeutic agent, for example a thrombolytic drug. For example, FIG. 7B shows a balloon 131 comprising micro-needles 135 capable of introducing a drug (the therapeutic intervention) to the treatment site. Such drug delivery balloon is configured such that at least some portion of the balloon 131 would come into contact with the blood vessel wall W upon the balloon's inflation. After the balloon is appropriately positioned as noted above, the balloon 131 is inflated to position the distance between the catheter body 100 and the blood vessel wall W substantially within the imaging depth of the side viewing mechanism 120. The drug is thereafter introduced into the balloon via lumen 133, allowing the drug to flow into the vessel wall W through the microneedles 135. Optionally, the drug comprises macromolecule carriers with predetermined drug release rates to be delivered to the vessel wall for therapy. The microneedles 135 could be solid needles or hollow needles. In case of solid needles, the microneedles 135 could be made of metals or bioerodible polymers. The bioerodible microneedles could then be coated or loaded with the desired therapeutic agent. If they are made of bioerodible polymers, the microneedles 135 could be embedded in the lesion and the drug could be delivered at the target site over a period of time as the microneedles 135 slowly erode.

Alternatively, to deliver the therapeutic agent into the blood vessel wall, the balloon 131 could comprise perforations and the therapeutic agent would seep out through the perforations. Such a drug delivery balloon could be a dual-lumen balloon comprising an inner and outer lumen with the therapeutic agent trapped between the inner and outer lumens. The inner lumen could be in fluid communication with the proximal end of the catheter, for example through lumen 133. The balloon may have micropores on its outer lumen surface. When fluid pressure is imparted on the therapeutic agent from the lumen of the inner balloon lumen, the pressure is forced onto the outer lumen, thereby forcing the therapeutic agent out of the micropores of the outer balloon. In another embodiment, the therapeutic intervention comprises a stent-delivery balloon configured to deliver a stent to a lesion. Alternatively, the therapeutic intervention comprises an angioplasty balloon. In another embodiment the therapeutic intervention comprises a thrombectomy device or an aspiration catheter to treat lesions such as thrombus.

While the above is a complete description of the preferred embodiments of the invention, various alternatives, modifications, and equivalents may be used. Therefore, the above description should not be taken as limiting the scope of the invention which is defined by the appended claims.

What is claimed is:

1. An angioscope for optically imaging a luminal wall, said angioscope comprising:
a tubular sheath having a proximal end, a distal end, a central lumen, and a light source disposed at the distal end to direct light axially from the sheath;
a central member reciprocatably received in the central lumen and having a proximal end and a distal end;
an image viewing element at the distal end of the central member; and
a lateral reflector disposed on the central member to reflect light from structures on the luminal wall illuminated by the light source to the image viewing element.

2. An angioscope as in claim 1, wherein the light source comprises at least one fiberoptic element disposed axially in a wall of the tubular sheath.

3. An angioscope as in claim 2, wherein the light source comprises a plurality of fiberoptic elements circumferentially spaced-apart in the wall of the tubular sheath.

4. An angioscope as in claim 3, wherein said plurality of fibers are spaced-apart over the entire circumference of the wall of the tubular sheath.

5. An angioscope as in claim 1, wherein the light source comprises a light emitting diode.

6. An angioscope as in claim 1, wherein the image viewing element comprises a fiberoptic bundle having a distal surface for receiving an image.

7. An angioscope as in claim 6, further comprising a red pass or yellow pass filter connected to receive light from the fiberoptic bundle.

8. An angioscope as in claim 1, wherein the image viewing element comprises a CCD at the distal end of the central member for receiving the image.

9. An angioscope as in claim 1, wherein the lateral reflector comprises a reflective element having a single flat reflective surface for reflecting images disposed laterally from the central member.

10. An angioscope as in claim 1, wherein the lateral reflector comprises a reflective element having multiple flat surfaces disposed to reflect images from a circumferential arc surrounding the central member.

11. An angioscope as in claim 10, wherein the reflective element comprises a prism with at least three reflective surfaces.

12. An angioscope as in claim 1, wherein the lateral reflective element comprises a partial or full conical prism to reflect a continuous image spanning a circumferential arc surrounding the central member.

13. An angioscope as in claim 10, wherein the circumferential arc extends fully around the central member.

14. An angioscope as in claim 1, further comprising a guidewire lumen.

15. An angioscope as in claim 14, wherein the guidewire lumen is disposed in the tubular sheath.

16. An angioscope as in claim 14, wherein the guidewire lumen is disposed along one side of a distal section of the sheath.

17. An angioscope as in claim 1, further comprising an inflatable occlusion member circumscribing a distal portion of the tubular sheath.

18. An angioscope as in claim 17, wherein the occlusion element is elastic and the tubular sheath has a plurality of inflation ports disposed to permit an infusion medium flowing through its central lumen to flow into and inflate the occlusion member.

19. A method for viewing the wall of a blood vessel, said method comprising:
introducing a tubular sheath into the blood vessel;
illuminating the wall of the blood vessel in an axial direction from a light source on a distal end of the sheath;
advancing a central member from a central lumen of the sheath;
reflecting an optical image of the vessel wall with a lateral reflector on the central member to an image viewing element on the central member; and
transmitting the optical image to a viewing screen to provide a real time image of the blood vessel wall.

20. A method as in claim 19, wherein illuminating comprises delivering light through optical fibers present in a wall of the tubular sheath.

21. A method as in claim 19, wherein illuminating comprises emitting light from one or more LEDs at the distal end of the tubular sheath.

22. A method as in claim 19, wherein the lateral reflector collects an image circumscribing at least 180° around the central member.

23. A method as in claim 22, wherein the image circumscribes 360° around the central member.

24. A method as in claim 19, wherein the viewing element comprises a fiberoptic bundle which carries the light image to a proximal end of the imaging core.

25. A method as in claim 24, wherein light from the fiberoptic bundle is selectively filtered to pass red light to enhance an image of thrombus.

26. A method as in claim 24, wherein light from the fiberoptic bundle is selectively filtered to pass yellow light to enhance an image of plaque.

27. A method as in claim 19, wherein the viewing element comprises a CCD camera which converts the image to an electronic signal and transmits the signal down the central member.

28. A method as in claim 20, further comprising inflating an occlusion member circumscribing a distal portion of the tubular sheath.

29. A method as in claim 28, wherein inflating comprises diverting a portion of an infusion medium flowing through the tubular sheath into the occlusion member, wherein the occlusion member comprises an elastic balloon which inflates when infusion medium flows through the sheath and which deflates and collapses over the sheath when infusion medium stops flowing through the sheath.

30. A device for viewing a delivery site within a body passageway and delivering a therapeutic intervention, the device comprising:
a catheter comprising a catheter body having a proximal end, a distal end, and first and second lumens there-between;
a delivery mechanism disposed at the distal end of the catheter body, wherein the delivery mechanism is configured to deliver a therapeutic intervention; and
a side-viewing optical scope disposed at the distal end, wherein the side-viewing scope allows determination of a delivery site for the therapeutic intervention;
wherein the first lumen comprises a plurality of optical fibers in optical communication with the side-viewing mechanism, and wherein the second lumen is in fluid communication with the delivery mechanism.

31. The device of claim 30, wherein the side-viewing mechanism comprises an imaging lens and a beam director.

32. The device of claim 31, wherein the beam director is a prism.

33. The device of claim 30, further comprising a third lumen in fluid communication with a flushing port, the flushing port located at or near the distal end of the catheter body.

34. The device of claim 30, wherein the optical fibers transmit electromagnetic radiation of a predetermined wavelength range, flowing bi-directionally between the proximal end and distal end of the catheter body.

35. The device of claim 34, wherein the wavelength range of the electromagnetic radiation is the visible light spectrum.

36. The device of claim 30, wherein the delivery mechanism is an angioplasty balloon.

37. The device of claim 30, wherein the delivery mechanism is a stent delivery mechanism.

38. The device of claim 30, wherein the delivery mechanism is a drug delivery balloon.

39. The device of claim 38, wherein at least some part of the balloon is configured to contact the body passageway when the balloon is inflated.

40. The device of claim 38, wherein the drug delivery balloon comprises one or more microneedles along an outer surface of the balloon.

41. The device of claim 40, wherein the microneedles facilitate drug delivery into a wall of the body passageway by allowing penetration of the drug into the wall.

42. The device of claim 40, wherein the drug comprises macromolecule carriers with predetermined release rates to be delivered to the body passageway.

43. The device of claim 38, wherein the drug delivery balloon comprises perforations along an outer surface of the balloon.

44. The device of claim 38, wherein the balloon is controllably inflated to position the distance between the catheter and the delivery site substantially within an imaging depth of field of an imaging lens.

45. The device of claim 38, wherein the inflatable balloon occupies an angular region around the catheter, the angular region comprising less than 360 degrees to accommodate a guidewire, where at least some portion of the guidewire is located exterior to the catheter.

46. The device of claim 38, wherein the balloon is substantially transparent.

47. The device of claim 30, wherein the delivery mechanism is configured to treat thrombus.

48. The device of claim 47, wherein the delivery mechanism includes a thrombus aspiration catheter, a thrombectomy device, or a device for delivering a thrombolytic agent.

49. A method of using a catheter for a body passageway, comprising:
advancing a multi-lumen catheter having a proximal end and a distal end into a body passageway, wherein the distal end of the catheter comprises a side-viewing mechanism and a delivery mechanism configured to deliver a therapeutic intervention to the body passageway, and wherein the side-viewing mechanism is in optical communication with the proximal end of the catheter and the delivery mechanism is in fluid communication with the proximal end of the catheter;
determining a site for delivery of the therapeutic intervention using the side-viewing mechanism; and
delivering a therapeutic intervention to the body passageway.

50. The method of claim 49, wherein the determining comprises determining a lesion site for delivery of the therapeutic intervention.

51. The method of claim 50, wherein the determining a lesion site comprises observing a location or distribution of the therapeutic intervention relative to the lesion site via the side viewing mechanism.
